(19) United States
(12) Patent Application Publication
(10) Pub. No.: US 2011/0305312 A1
(43) Pub. Date: Dec. 15, 2011

(54) X-RAY SOURCE COMPRISING A FIELD EMISSION CATHODE

(75) Inventor: Qui-Hong HU, GÖTEBORG (SE)

(73) Assignee: LIGHTLAB SWEDEN AB, SALTSJÖBADEN (SE)

(21) Appl. No.: 13/141,941

(22) PCT Filed: Feb. 8, 2010
(86) PCT No.: PCT/EP2010/051481

§ 371 Date: Aug. 31, 2011

(30) Foreign Application Priority Data
Feb. 18, 2009 (EP) 09153101.2

(51) Int. Cl. H01J 35/06; A61B 6/03; G01N 23/04

(52) U.S. Cl. 378/4; 378/122; 378/62

(57) ABSTRACT

The present invention relates to an x-ray source, comprising a field emission cathode, an anode, connectors for allowing application of a high voltage between the cathode and the anode for enabling emission of an x-ray beam, and an evacuated chamber inside of which the anode and the cathode are arranged, the evacuated chamber having an x-ray transparent window, wherein the field emission cathode consists of a carbonized solid compound foam having a continuous cellular structure, the continuous cellular structure providing multiple emission cites for emission of electrons onto the anode when the high voltage is applied. The field emission cathode provides for the possibility to increase the efficiency of the x-ray system as it is possible to in a much higher degree control the electrons emitted by the field emission cathode in terms of switching time, current, kinetic energy and the emission direction.

### FIELD OF THE INVENTION

The present invention relates to an x-ray source comprising a field emission cathode. The present invention also relates to a method for scanning an object using a field emission based x-ray source.

### DESCRIPTION OF THE RELATED ART

Systems for generation of x-ray radiation are used, for example, in medical diagnostics in order to acquire radiographic images or to produce planar images for technical diagnostic applications. In the field of technical diagnostic imaging, x-rays are especially effective at penetrating internal structures of a solid object to be examined, and the images formed by the x-rays that pass there through reveal internal flaws or structural defects of the object. Technical diagnostic x-ray imaging thus provides a valuable quality control inspection tool for evaluating structural aspects of a product during manufacture and over the useful life of the product. This form of diagnostic analysis is advantageous over other types of evaluation, since the imaging object need not be destroyed in the process of the evaluation. For this reason, technical diagnostic imaging is also known as non-destructive testing.

An x-ray tube for technical imaging applications typically comprises an electron gun having a cathode that is excited to emit a beam of electrons that are accelerated to an anode. The cathode is generally based on thermionic emission, and the anode may be comprised of a metal target surface, such as tungsten, from which x-rays are generated due to the impact of the accelerated electrons. By disposing the anode surface at an angle to the axis of the electron beam, the x-rays may be transmitted in a direction generally perpendicular to the electron beam axis.

The x-rays may then be passed through a beryllium window used to provide a vacuum seal within the x-ray tube. Thereafter, the x-rays exit the x-ray tube along a generally conical path where the apex of the cone is roughly coincident with the spot on target formed by the impinging electron beam.

The use of x-ray tubes based on thermionic emission however provides limited control possibilities, especially due to the fact that such x-ray tubes exhibit a slow reaction time, high energy consumption, and have a high space requirement. Such x-ray tubes are therefore less suited for the modern applications.

An approach has been made to solve the above mentioned problems by replacing the thermionic emission cathode with a field emission cathode. An example of such an implementation is disclosed in US 2006/0039532, in which the field-emission cathode constitutes of an array of sharp points that emits electrons when a small electric potential is placed between the tip and an extraction electrode. The sharp tips enhance the field-emission effect since a relatively small voltage creates a large electric field at each point, allowing electrons to tunnel from the tip into the vacuum.

However, the use of such a field emission cathode provides a limiting result as a large extraction current, giving high energy consumption, is needed to achieve a steady emission of electrons. Thus, due to the high energy consumption such an implementation is undesirable as it limits the mobility of the resulting x-ray system.

There is therefore a need for an improved x-ray system that at least alleviates the prior art reliability problems.

### SUMMARY OF THE INVENTION

According to an aspect of the invention, the above is met by an x-ray source, comprising a field emission cathode, an anode, a connector for allowing application of a high voltage potential between the cathode and the anode for enabling emission of an x-ray beam, and an evacuated chamber inside of which the anode and the cathode are arranged, wherein the field emission cathode consists of a carbonized solid compound foam having a continuous cellular structure, the continuous cellular structure providing multiple emission cites for emission of electrons in the direction of the anode when the high voltage potential is applied.

The general concept of the present invention is based on the fact that it is possible to in a more accurate way control the emission of electrons, in the direction from the cathode to the anode, such that only an adequate amount of x-ray is emitted. By using a field emission cathode instead of a prior art electron beam source (e.g. “filament” or thermionic emission cathode), it is possible to increase the efficiency of the x-ray source as it is possible to in a much higher degree control the electrons emitted by the field emission cathode in terms of switching time, current, kinetic energy and the emission direction. Furthermore, a very fast reaction time for the electron emission (and thus also for the x-ray emission) is thereby achieved, thereby providing for an x-ray source having stable x-ray output characteristics. Additionally, the possibility to provide very sharp tips, resulting from the carbonized solid foam, in combination with the large plurality of emission sites also allows for increased efficiency of the x-ray source. Furthermore, the x-ray source according to the invention also have the ability to produce focused electron beams with a small energy spread that can potentially enable ultrafine focal spots for high-resolution imaging.

The evacuated chamber preferably has a pressure of approximately 10⁻⁴ Pa or lower for allowing free flow of the emitted electrons. Due to the use of the inventive concept of introducing a field emission cathode based on a continuous cellular structure it may however be possible to decrease the requirement of a high vacuum, thus making the x-ray source according to the invention easier to manufacture.

According to a preferred embodiment of the invention, the carbonized solid compound foam is transformed from a liquid compound comprising a phenolic resin and at least one of a metal salt, a metal oxide.

Preferably, the evacuated chamber may be of glass or metal. In case of using a metal chamber, the chamber may have an x-ray transparent window. The window, may for example be of Beryllium thereby providing for a controlled emission of the x-ray out from of the x-ray source.

Preferably, the x-ray source further comprises a cooling mechanism for cooling the anode, such as a metal anode. A decrease in the temperature at the anode further enhances the x-ray emission.

In an alternative embodiment, the x-ray source further comprising a focusing electrode for focusing electrons emitted by the field emission cathode. Also, the x-ray source may alternatively further comprise an extraction electrode for extracting electrons in a direction from the cathode to the anode, thereby forming a triode structure. Additionally, the x-ray source may also or instead comprise a plurality of controllable field emission cathodes. By means of using a plurality of field emission cathodes it may be possible to allow for a pixel based x-ray emission, increasing the flexibility when steering the x-ray emission to a specific reception site. Advantageously, the x-ray source may be adapted for generating a spectrum peak for the x-beam at approximately 20 keV when providing a current of as low as 1 mA. Accordingly, a suitable x-ray source may be provided with only small energy consumption, thus making the x-ray source more mobile.

From a system perspective it is possible to form an x-ray system by including an x-ray source as discussed above together with an x-ray detector, an object holder for receiving an object to be imaged, the object holder arranged between the x-ray transparent window and the x-ray detector, and a control unit for controlling the x-ray emission and for collecting data from the x-ray detector. Preferably, the object holder is rotatable by means of the control unit, thereby allowing data collection of the object from different viewing angles. The system may also comprise a dosage sensor for detecting an X-ray dosage generated by the x-ray source, wherein the control unit is adapted to receive dosage information from the dosage sensor for controlling the x-ray system. A further controllable system may thereby be provided.

Preferably, such an x-ray system is portable and thus may comprise a battery operated, high voltage power supply, advantageously allowing the x-system to be mobile for field applications.

According to another aspect of the invention there is provided a method for scanning an object, the method comprising the steps of providing an x-ray source, comprising a field emission cathode, an anode, a connector for allowing application of a high voltage between the cathode and the anode for enabling emission of an x-ray beam, and an evacuated chamber inside of which the anode and the cathode are arranged, wherein the field emission cathode consists of a carbonized solid compound foam having a continuous cellular structure, the continuous cellular structure providing multiple emission cites for emission of electrons in the direction of the anode when the high voltage potential is applied, positioning an object in a path for intercepting at least one x-ray beam emitted by the anode, activating the x-ray source by means of a control unit such that x-ray beams are emitted by the anode, detecting x-ray intensities by means of an x-ray detector, and generating image data using the control unit based on the detected x-ray intensities. The method may also comprise the step of generating data for constructing a three-dimensional image of the object.

This aspect of the invention provides similar advantages as according to the above discussed x-ray source and system, including for example increase efficiency and portability.

### BRIEF DESCRIPTION OF THE DRAWINGS

These and other aspects of the present invention will now be described in more detail, with reference to the appended drawings showing currently preferred embodiments of the invention, in which:

FIG. 1 is a conceptual view of a first embodiment of an x-ray source according to the present invention;

FIG. 2 is a conceptual view of a second embodiment of an x-ray source according to the present invention;

FIG. 3 is a conceptual view of an x-ray system according to a currently preferred embodiment of the present invention; and

FIG. 4 is an x-ray emission spectrum illustrating the relation between applied energy and emitted x-ray.

### DETAILED DESCRIPTION OF CURRENTLY PREFERRED EMBODIMENTS

The present invention will now be described more fully hereinafter with reference to the accompanying drawings, in which currently preferred embodiments of the invention are shown. This invention may, however, be embodied in many different forms and should not be construed as limited to the embodiments set forth herein; rather, these embodiments are provided for thoroughness and completeness, and fully convey the scope of the invention to the skilled addressee. Like reference characters refer to like elements throughout.

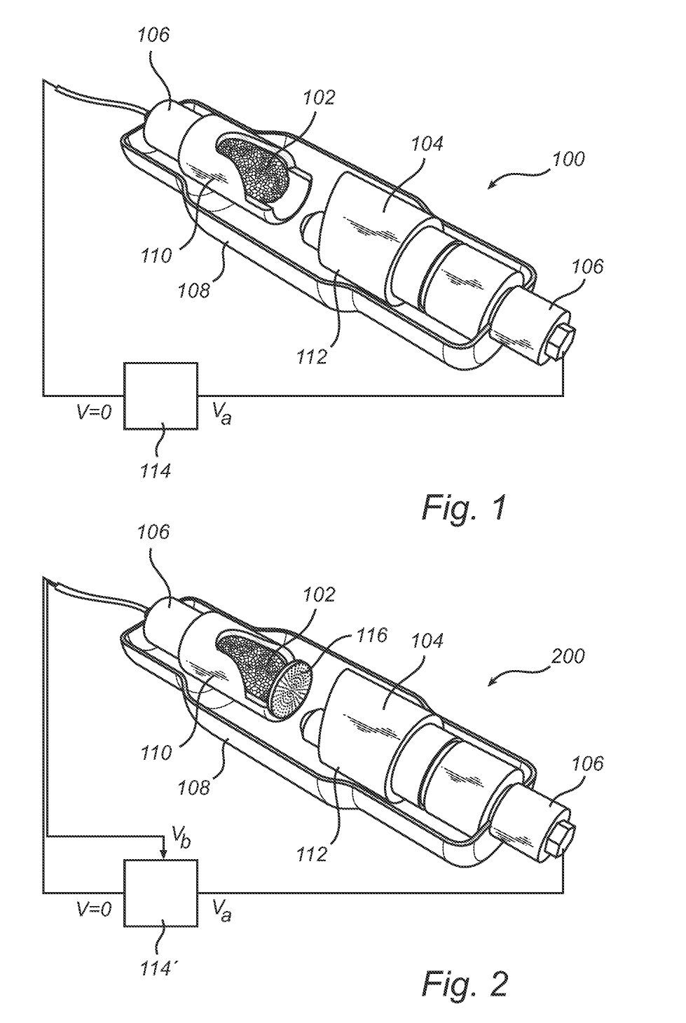

Referring now to the drawings and to FIG. 1 in particular, there is depicted a conceptual view of a first embodiment of an x-ray source 100 according to the present invention. The x-ray source 100 comprises a cathode and an anode (i.e. diode structure), where the cathode is a field emission cathode 102 and the anode preferably is a metal anode 104, for example of copper. Each of the cathode 102 and the anode 104 are provided with an electrical connector 106 which extends out of an evacuated chamber 108, for example of glass or metal, and at least having a window transparent to x-rays when the chamber is of metal. The chamber 108 preferably has a pressure around approximately 10⁻⁴ Pa, but could of course depending of the application be more or less. For connecting the cathode 102 and the anode 104 to the electrical connectors 106, each of the cathode 102 and the anode 104 are provided with a holder 110 and 112, respectively.

The field emission cathode 102 preferably consists of a carbonized solid compound foam having a continuous cellular structure, the continuous cellular structure providing multiple emission cites for emission of electrons in the direction towards the anode when the high voltage is applied. Possibly, the carbonized solid compound foam may be transformed from a liquid compound comprising a phenolic resin and at least one of a metal salt, a metal oxide.

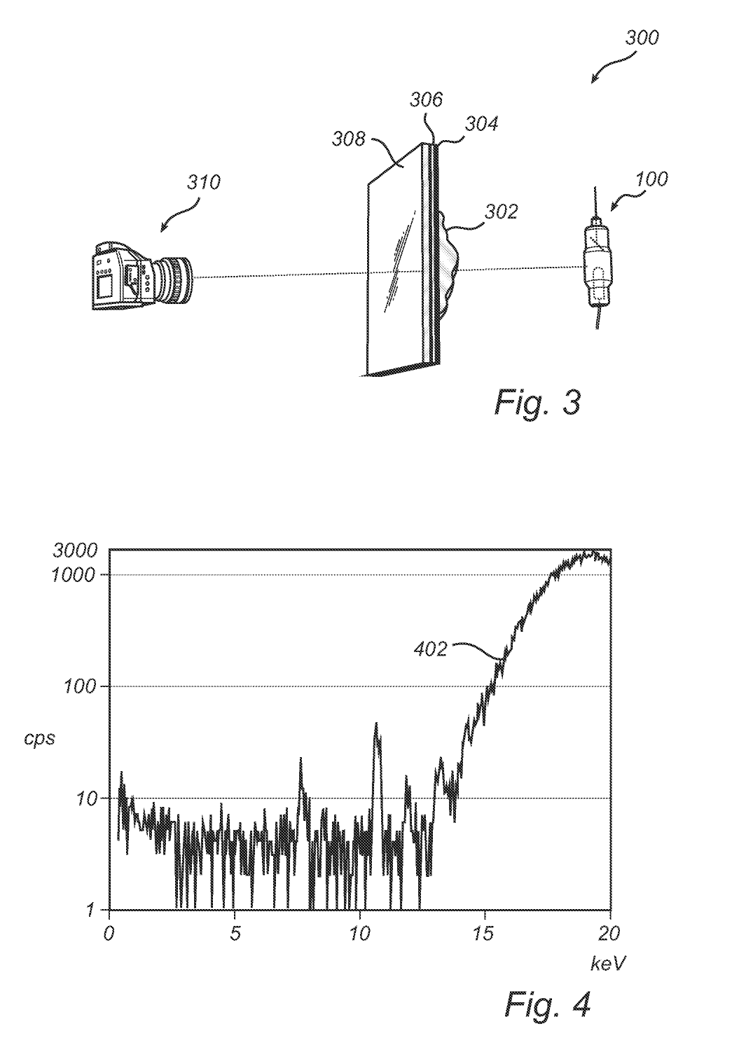

By means of the continuous cellular structure, a large plurality of emission sites may be provided, each having very sharp tips, thereby allowing for high emission efficiency. Consequently, when applying a high voltage to the respective connectors 106, electrons are emitted from the cathode when the electrical field exceeds a threshold field for emission. For providing such a high voltage, a power supply 114 may be used. The power supply may be portable, including for example a power source such as a battery or similar. Additionally, as can be seen in FIG. 1, the anode 104 surface may be disposed at an angle to the axis of the electron beam such that x-rays may be transmitted in a direction generally perpendicular to the electron beam axis. A further description relating to this is provided in relation to FIG. 3.

Turning now to FIG. 2, illustrating a conceptual view of a second embodiment of an x-ray source 200 according to the present invention. The x-ray source 200 is essentially similar to the x-ray source 100 of FIG. 1, having a difference in that the x-ray source 200 is of a triode structure, i.e. also comprising a gate electrode 116 arranged at a distance from the field emission cathode 102, preferably in the range of a few tens of micrometers to several millimeters from the surface of the cathode 102 surface. By means of the applying a bias field between gate electrode 116 and the cathode 102, it may be possible to increase the extractions of electrons in the direction of the anode 104. For such an operation, the gate electrode 116 may be connected, through the connector 106, to a slightly modified power supply 114, thereby allowing for the application of a bias voltage for the gate electrode 116. By mean of the triode structure it will be possible to independently at least adjust the current intensity and kinetic energy of the x-ray source 200. The cathode structure can also provide a fine beam focus, which is advantageous in relation to the emission of x-ray. Accordingly, geometrical parameters of the gate electrode 116 may be optimized based on the specific application, including for example different types of gate electrode shapes comprising for example a grid mesh design, including adjustment of parameters relating to mesh wire thickness and mesh opening area.

In FIG. 3 it can be seen a conceptual view of an x-ray system 300 for scanning an object 302 according to a currently preferred embodiment of the present invention, comprising a field emission based x-ray source 100 as is disclosed in FIG. 1. The x-ray source may also be an x-ray source 200 as is disclosed in FIG. 2. In both cases, the x-ray source may comprise a cooling mechanism, when necessary, for cooling the anode, which may get warm under the electron excitation.

The x-ray system 300 also comprises an x-ray detector, for example comprising a surface 304 for receiving the object 302, a fluorescent screen 306, a lead glass 308 and a digital camera 310. Additionally, the x-ray system 300 may include a control unit (not shown) for controlling the operation of the x-ray system 300. Also, a dosage sensor (not shown) may also be provided for detecting an x-ray dosage generated by the x-ray source 100. It should be noted that other types of x-ray detectors may be used and are within the scope of the present invention, including for example photographic plates, photostimulable phosphors (PSPs), different types of Geiger counters, scintillators, and direct semiconductor detectors. Additional detectors may also possibly be used.

For positioning the object, an object holder may be provided, where the object holder may be controlled, for example by the control unit, for rotation and or multi-direction displacement of the object. By collecting imaging data of the object from different angles it may be possible to generate a three-dimensional x-ray image of the object.

During operation of the x-ray system 300, the object 302 is positioned in a path for intercepting an x-ray beam emitted by the anode 104 of the x-ray source. Thereafter, the control unit activates the x-ray source 100 such that x-ray beams are emitted from the anode. The x-ray detector is also activated, and provides detection of x-ray intensities resulting from the x-ray beam and its interception with the object 302. Thereafter, the control unit, or a separate computing device, may generate image data based on the detected x-ray intensities.

Turning finally to FIG. 4 which is an x-ray emission curve illustrating the relation between applied energy and emitted x-ray for an x-ray beam emitted by the x-ray source 100. The emission spectrum 402 exhibits a peak at around 20 keV with a current of less than 1 mA, indicating a high efficiency of the x-ray source 100. Prior art x-ray sources comprising for example filament or thermionic emission cathode elements may have to be applied with an control current at a much higher level for reaching such an output.

The executable instructions of a computer program for controlling the as shown x-ray system can be embodied in any computer readable medium for use by or in connection with an instruction execution system, apparatus, or device, such as a computer based system, processor containing system, or other system that can fetch the instructions from the instruction execution system, apparatus, or device and execute the instructions.

As used here, a “computer readable medium” can be any means that can contain, store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium, such as a removable storage device. More specific examples (a non exhaustive list) of the computer readable medium can include the following: an electrical connection having one or more wires, a portable computer diskette, a random access memory (RAM), a read only memory (ROM), an erasable programmable read only memory (EPROM or Flash memory), an optical fibre, and a portable compact disc read only memory (CDROM).

Furthermore, the skilled addressee realizes that the present invention by no means is limited to the preferred embodiments described above. On the contrary, many modifications and variations are possible within the scope of the appended claims. For example, both the x-ray sources illustrated in FIGS. 1 and 2 may be arranged to include a plurality of controllable field emission cathodes, thereby providing for the possibility to emit pixel based x-ray beams in the configurations of, apart from the most common single cathode to single anode, either multi-cathode to single anode or single cathode to multi-anode, and even to facetted multi-anode.

We claim:

1. An x-ray source, comprising:
a field emission cathode;
an anode;
a connector for allowing application of a high voltage potential between the cathode and the anode for enabling emission of an x-ray beam, and
an evacuated chamber inside of which the anode and the cathode are arranged;
characterized in that field emission cathode consists of a carbonized solid compound foam having a continuous cellular structure, the continuous cellular structure providing multiple emission cites for emission of electrons onto the anode when the high voltage is applied.

2. X-ray source according to claim 1, wherein the evacuated chamber has a pressure of 10⁻⁴ Pa or lower.

3. X-ray source according to claim 1, wherein the carbonized solid compound foam is transformed from a liquid compound comprising a phenolic resin and at least one of a metal salt, a metal oxide.

4. X-ray source according to claim 1, wherein the evacuated chamber is of glass or metal having an x-ray transparent window.

5. X-ray source according to claim 1, further comprising a cooling mechanism for cooling the anode.

6. X-ray source according to claim 1, further comprising a focusing electrode for focusing electrons emitted by the field emission cathode.

7. X-ray source according to claim 1, further comprising an extraction electrode, wherein the anode, the cathode and the extraction electrode together forms a triode structure.

8. X-ray source according to claim 1, comprising a plurality of controllable field emission cathodes.

9. X-ray source according to claim 1, generating a spectrum peak for the x-beam at approximately 20 keV when providing a current of less than 1 mA.

10. An X-ray system, comprising:
an x-ray source according to claim 1;
an x-ray detector;
an object holder for receiving an object to be scanned, the object holder arranged between the x-ray transparent window and the x-ray detector, and
a control unit for controlling the x-ray emission and for collecting data from the x-ray detector.

11. X-ray system according to claim 10, wherein position of the object holder is rotatable by means of the control unit, thereby allowing data collection of the object from different viewing angles.

12. X-ray system according to claim 10, further comprising a dosage sensor for detecting an x-ray dosage generated by the x-ray source, and wherein the control unit is adapted to receive dosage information from the dosage sensor for controlling the x-ray system.

13. X-ray system according to claim 10, wherein the x-ray system comprises a battery power source and is portable.

14. A method for scanning an object, the method comprising the steps of:
providing an x-ray source, comprising a field emission cathode, an anode, connectors for allowing application of a high voltage between the cathode and the anode for enabling emission of an x-ray beam, and an evacuated chamber inside of which the anode and the cathode are arranged, wherein the field emission cathode consists of a carbonized solid compound foam having a continuous cellular structure, the continuous cellular structure providing multiple emission cites for emission of electrons in the direction of the anode when the high voltage is applied;
positioning an object in a path for intercepting at least one x-ray beam emitted by the anode;
activating the x-ray source by means of a control unit such that x-ray beams are transmitted by the anode;
detecting x-ray intensities by means of an x-ray detector; and
generating image data using the control unit based on the detected x-ray intensities.

15. Method according to claim 14, further comprising the step of generating data for constructing a three-dimensional image of the object.

16. X-ray system according to claim 11, further comprising a dosage sensor for detecting an x-ray dosage generated by the x-ray source, and wherein the control unit is adapted to receive dosage information from the dosage sensor for controlling the x-ray system.

17. X-ray system according to claim 11, wherein the x-ray system comprises a battery power source and is portable.

18. X-ray system according to claim 12, wherein the x-ray system comprises a battery power source and is portable.
